(19) United States
(12) Patent Application Publication
(10) Pub. No.: US 2024/0246325 A1
(43) Pub. Date: Jul. 25, 2024

(54) DURABLE FUNCTIONAL AND AESTHETIC LAMINATES

(71) Applicant: BIXBY INTERNATIONAL CORPORATION, Newburyport, MA (US)

(72) Inventors: Marc GAGNON, Exeter, NH (US); Timothy A. LIBERT, Hockessin, DE (US); Rick BASS, Freemont, NH (US)

(73) Assignee: BIXBY INTERNATIONAL CORPORATION, Newburyport, MA (US)

(21) Appl. No.: 18/566,066

(22) PCT Filed: Jun. 16, 2022
(86) PCT No.: PCT/US2022/033851

§ 371 Date: Nov. 30, 2023

Related U.S. Application Data
(60) Provisional application No. 63/212,248, filed on Jun. 18, 2021.

(51) Int. Cl. B32B 27/08; A47B 37/04; A47C 31/00; A61L 27/60; B32B 3/30; B32B 7/12; B32B 27/18; B32B 27/36; B32B 27/40; B44C 5/04; B60N 3/04; B60R 13/01; E04F 15/10

(52) U.S. Cl. CPC B32B 27/08; A47B 37/04; A47C 31/004; B32B 3/30; B32B 7/12; B32B 27/18; B32B 27/36; B32B 27/40; A61L 27/60; B32B 2255/10; B32B 2255/26; B32B 2274/00; B32B 2307/4026; B32B 2307/422; B32B 2307/558; B32B 2307/584; B32B 2307/71; B32B 2307/712; B32B 2307/714; B32B 2307/7376; B32B 2307/738; B32B 2419/00; B44C 5/04; B60N 3/04; B60R 13/011; E04F 15/107

(57) ABSTRACT

Laminate coverings having multiple layers with different functionalities for various applications including prosthetic, outdoor furniture and automotive cargo/trunk liner and floor mat applications. In some instances, the laminate coverings may also be used as decorative skins or be used in other applications for its decorative or aesthetic properties, the laminate coverings having improved UV, weatherability, scratch and mar resistance, among other enhancements.

### CROSS REFERENCE TO RELATED APPLICATIONS

This application claims the benefit of U.S. Provisional Patent Application No. 63/212,248 filed Jun. 18, 2021, which is incorporated herein by reference.

### FIELD OF THE INVENTION

The present invention relates to laminate coverings, and more specifically, to durable multi-purpose laminate coverings having multiple layers with different functionalities and decorative aesthetics.

### BACKGROUND OF THE INVENTION

Laminates can come in different forms and have different utilities and applications. In some instances, laminates are generally used to cover floors, walls or other surfaces in need of protective coverings and decoration. In other instances, laminates are used as floor coverings and commonly installed in residential and commercial spaces to provide functionalities such as durability, stain resistance and aesthetic or fashion characteristics.

Laminate flooring was invented around 1977 by the Swedish company Perstorp, and sold under the brand name Pergo. The company first marketed its product in Europe in 1984, and later to the United States in 1994. Pergo is the most widely known laminate flooring manufacturer, but the trademark Pergo is not synonymous for all laminate floors.

Glueless laminate flooring was invented in 1996 by the Swedish company Välinge Aluminium (now Välinge Innovation) and sold under the names of Alloc and Fiboloc. However, a system for holding flooring panels together was also developed in parallel by the Belgian company Unilin, which released it in 1997 and sold it under the name of Quick-Step flooring.

Laminate flooring is a multi-layer synthetic flooring product fused together with a lamination process. Laminate flooring simulates wood (or sometimes stone) with a photographic applique layer under a clear protective layer. The inner core layer is usually composed of melamine resin and fiber board materials.

Laminate flooring has grown significantly in popularity, perhaps because it may be easier to install and maintain than more traditional surfaces such as hardwood flooring. It may also have the advantages of lower cost and require less skill to install than alternative flooring materials. It is also reasonably durable, hygienic (several brands contain an antimicrobial resin), and relatively easy to maintain.

Methods for printing decorative patterns in conventional decorative floor coverings include directly printing patterns on a polyvinyl chloride resin sheet or on a substrate layer comprising glass fiber, and transferring printed patterns on paper or polyester film to a polyvinyl chloride resin sheet surface. However, these methods generally fail to produce natural patterns for having artificial traces. Particularly, although the characteristics of a pattern are superior when they are printed on paper or polyester transfer paper, the pattern itself changes due to the transfer process which in turn decreases pattern realism. Furthermore, although fillers are added to a polyvinyl chloride resin sheet layer in order to address surface irregularities that adversely affect pattern transfer, the fillers themselves can cause deterioration of the transferred patterns.

Secondly, conventional decorative floor coverings, in which an olefin resin skin layer such as a transparent polyvinyl chloride resin or polyethylene, is used on a printing layer so as to protect the printing layer, tend to generate microscopic air pockets during processing, so a yellowing phenomenon can occur due to the heat changes during processing. In addition, a certain thickness should be maintained in order to provide durability during use, and this thickness can result in further deterioration of transparency.

Thirdly, polyvinyl chloride resin has a low softening point of 80° C. to 100° C., and therefore can exhibit poor resistance to heat sources over 100° C. Although there have been cases where a non-foaming polyvinyl chloride resin or other materials have been used as a skin layer to improve heat resistance, problems of heat resistance have not been fundamentally solved.

Furthermore, a non-foaming polyvinyl chloride resin layer has been used under a conventional decorative floor covering as a balance layer, but this creates problems later on during installation of the flooring since the product weight is increased due to the resin layer.

While a variety of laminates are available, there still are no laminates where the laminate material includes multiple layers that are generally non-compatible with each other, but each provides its own unique properties such as durability, decorative functions and the like. In addition, the laminates currently available are not thermoformable, which limits their usability in applications such as prosthetic sockets, furniture, RV's, ATV's, boats, and curved flooring, to name a few.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

Keeping the continued need for better laminates in mind, the instant invention provides a laminate that incorporates certain performance attributes such as weather resistance, fade resistance, and scratch resistance along with certain aesthetic attributes such as the look of natural woods or stones, or any desired color, or any graphic such as logos or photographs. Some layers of this laminate product could also provide additional functionality such as thermochromism, cushioning, or luminescence, among other functions.

The instant invention also provides sandwiched laminates and processes for “sandwiching” different materials or different layers which are generally considered non-compatible with each other in order to enable laminate products described above. In its broadest aspects, the instant invention provides a laminate covering comprising multiple layers, said multiple layers selected from: (i) a functional surface layer; (ii) one or more functional layer(s); (iii) at least one tie layer; and (iv) a structural layer. The present invention also discloses the use of such laminates in prosthetic, outdoor furniture and automotive cargo, trunk liner and floor mat skin applications.

The instant invention in its broadest aspect provides a laminate covering comprising multiple layers, said multiple layers selected from: (i) a surface layer; (ii) one or more functional layer(s); (iii) at least one tie layer; and (iv) a structural layer.

One embodiment provides a laminate wherein the surface layer is durable and chemical resistant, UV resistant, moisture resistant, scratch resistant, and has outdoor weatherability as its primary function. Another embodiment provides a laminate where the surface layer includes an ionomer, ionomer blend, single cation ionomer, mixed cation ionomer, non-ionomer, or a mixture thereof. A further embodiment provides a laminate where the ionomer is selected from copolymers of ethylene with acrylic or methacrylic comonomers and optionally with alkyl acrylate and methacryalate ter-monomer, and the non-ionomer is selected from polyurethanes, polyurethane blends, polyamides, acrylics, acrylic blends, polyester, polyester blends, polyester copolymers, and polyester copolymer blends.

Another embodiment provides a laminate where the tie layer includes an olefinic, non-olefinic, or a thermoplastic adhesive layer, or combinations thereof. A further embodiment provides a laminate where the tie layer further includes grafted homo or co-polymers of ethylene, propylene, or butene, each optionally mixed with maleic anhydride, maleic acid, or glycidyl methacrylates.

Another aspect of the invention provides a laminate where the surface layer and/or the intermediate layer(s) is at least one of luminescent, thermochromic, is capable of displaying aesthetic effects, textured look, optics enabled, antistatic, has low coefficient of friction, or high coefficient of friction.

Yet another aspect of the instant invention provides a laminate where the surface layer includes additives to improve scratch resistance, impact resistance, moisture resistance, and outdoor weatherability, where the additives are selected from a non-silica hardener, siloxanes, amine and phenolic light stabilizers, and impact modifiers such as copolymers of styrene-butadiene rubbers and acrylonitrile-butadiene-styrene copolymers.

In some embodiments, the total thickness of the laminate can be in the range of from about 0.1 mm to about 5.0 mm, or from about 0.1 mm to about 5.0 cm, or from about 0.5 mm to about 5.0 mm, or from about 0.5 mm to about 5.0 cm.

In some embodiments, the thickness of the surface layer can be in the range of from about 0.1 mil to about 20 mils, or from about 0.1 micron to about 100 microns.

In some embodiments, the thickness of the functional layer can be in the range of from about 1 mil to about 25 mils, or from about 2 mils to about 25 mils, or from about 5 mils to about 25 mils.

An embodiment provides a laminate where the aesthetic effect is selected from fluorescence, pearlescence, printed coating, stone look, and wood look.

Another embodiment provides a laminate where the functional layer includes at least one of polyurethanes, polyamides, polyacrylates, polyureas, polyimides, olefinic copolymers, terpolymers, and functionalized olefins.

Another embodiment provides a laminate where the tie layer includes adhesives selected from the group consisting of homo or copolymers or terpolymers of olefinic polymers grafted with maleic anhydride, or glycidyl acrylate or methacrylate or amine groups or polyurethanes or polyamides or epoxy resins.

In some embodiments, the laminates disclosed herein are thermoformable to provide additional flexibility and usability for applications such as those having curved surfaces, e.g., curved flooring, laminates in vehicles, among others.

In one embodiment, a laminate covering includes a functional layer, a surface layer having additives, where the surface layer includes at least one of ionomer, ionomer blend, single cation ionomer, mixed cation ionomer, non-ionomer, and mixtures thereof, and the additives include at least one of non-silica hardener, siloxanes, amine and phenolic light stabilizers, and impact modifiers including copolymers of styrene-butadiene rubbers and acrylonitrile-butadiene-styrene copolymers. In this embodiment, the laminate covering also includes a tie layer between the functional layer and the surface layer.

In one embodiment, the functional layer is selected from the group consisting of polyurethanes, polyamides, polyacrylates, polyureas, polyimides, olefinic copolymers, terpolymers, functionalized olefins, and combinations thereof.

In one embodiment, the ionomer is selected from the group consisting of copolymers of ethylene with acrylic and methacrylic co-monomers optionally with alkyl acrylate and methacrylate ter-monomer, and combinations thereof, and where the non-ionomer is selected from the group consisting of polyurethanes, polyurethane blends, polyamides, acrylics, acrylic blends, polyester, polyester blends, polyester copolymers, polyester copolymer blends, and combinations thereof.

In one embodiment, the tie layer includes at least one of grafted homo or co-polymers of ethylene, propylene, or butene, each optionally mixed with maleic anhydride, maleic acid, or glycidyl methacrylate.

In one embodiment, the tie layer includes at least one of olefinic, non-olefinic, thermoplastic adhesive, and combinations thereof.

In one embodiment, the thermoplastic adhesive is selected from the group consisting of homo or copolymers or terpolymers of olefinic polymers grafted with maleic anhydride or glycidyl acrylate or methacrylate or amine groups or polyurethanes or polyamides or epoxy resins.

In one embodiment, the thickness of the functional layer is in the range of from about 1 mil to about 25 mils, the thickness of the surface layer is in the range of from about 0.1 mil to about 20 mils, and the thickness of the laminate covering is in the range of from about 0.1 mm to about 5 mm.

In some embodiments, the surface layer is configured to exhibit at least one of the following properties: luminescence, thermochromism, textured look, optics enabled, and anti-static, and the surface layer is configured to provide an aesthetic effect, the aesthetic effect selected from at least one of fluorescence, pearlescence, stone-look appearance and wood-look appearance.

In some embodiments, the surface layer is durable, thermoformable, chemical resistant, UV resistant, moisture resistant, impact resistant, scratch resistant, and has outdoor weatherability and soft feel.

In one embodiment, a process of manufacturing the laminate coverings disclosed herein includes tandem extrusion, coextrusion, belt lamination, roll coating and hot melt lamination.

In one embodiment, disclosed is a laminate having (i) an inner layer having a design, (ii) an outer layer having additives, the outer layer including at least one of ionomer, ionomer blend, single cation ionomer, mixed cation ionomer, non-ionomer, and mixtures thereof, and the additives comprising at least one of non-silica hardener, siloxanes, amine and phenolic light stabilizers, and impact modifiers including copolymers of styrene-butadiene rubbers and acrylonitrile-butadiene-styrene copolymers, (iii) a tie layer between the inner layer and the outer layer, and (iv) a structural layer configured to support (i), (ii) and (iii).

In one embodiment, the inner layer further includes a plurality of layers, where the plurality of layers is selected from the group consisting of a graphic carrier film layer having a decorative printed graphics, and a thermal insulator layer selected from at least one of thermoplastic urethane with fillers, blowing agents, luminescent additives, and combinations thereof.

In one embodiment, the outer layer further includes a plurality of layers, where the plurality of layers is selected from the group consisting of: (i) a topcoat of moisture curable aliphatic thermoplastic urethane; (ii) an embossed pattern layer; (iii) a clear layer of aliphatic thermoplastic urethane; or (iv) a combination of (ii) and (iii).

In one embodiment, the tie layer includes hot melt or moisture cured polyurethane. In another embodiment, the structural layer is an injection molded structural limb socket formed of a low temperature reinforced thermoplastic polyester (PET) compound.

In one embodiment, the inner layer further includes a plurality of layers, where the plurality of layers is selected from the group consisting of: a graphic carrier film layer having a decorative printed graphics, a color pigmented maleic anhydride grafted ethylene methyl acrylate (EMA) copolymer, and a thermal insulator layer selected from at least one of ionomer with fillers, blowing agents, luminescent additives, and combinations thereof.

In one embodiment, the outer layer further includes a plurality of layers, where the plurality of layers is selected from the group consisting of: (i) a topcoat of moisture curable aliphatic thermoplastic urethane, (ii) an embossed pattern layer, (iii) a layer of ionomer elastomer, (iv) a clear maleated functional olefin hot melt layer, (v) a skeleton print foil with wood grain layer, or (vi) a combination of (ii), (iii), (iv) and (v).

In one embodiment, the tie layer includes hot melt maleated olefin homo and copolymers. In some embodiments, the structural layer is selected from the group consisting of wood composite boards and plastic wood composites. In some embodiments, the outer layer is durable, thermoformable, chemical resistant, UV resistant, moisture resistant, impact resistant, scratch resistant, and has outdoor weatherability and soft feel.

In one embodiment, a laminate includes a structural layer, an inner layer having a design, first tie layer between the structural layer and the inner layer, an outer layer having additives, the outer layer having at least one of ionomer, ionomer blend, single cation ionomer, mixed cation ionomer, non-ionomer, and mixtures thereof, and the additives including at least one of non-silica hardener, siloxanes, amine and phenolic light stabilizers, and impact modifiers including copolymers of styrene-butadiene rubbers and acrylonitrile-butadiene-styrene copolymers, and second tie layer between the inner layer and the outer layer.

In one embodiment, the inner layer further includes a plurality of layers, where the plurality of layers is selected from the group consisting of: a graphic carrier film layer having a decorative printed graphics, and a decorative layer having one or more ionomers with solid color pigment.

In one embodiment, the outer layer further includes a plurality of layers, where the plurality of layers is selected from the group consisting of: (i) a topcoat of moisture curable aliphatic thermoplastic urethane, (ii) an embossed pattern layer, (iii) a layer selected from the group consisting of ionomer elastomer, nano-silica and pearlescent pigment, or (iv) a combination of (ii) and (iii).

In one embodiment, each of the first tie layer and the second tie layer includes maleic anhydride grafted functional olefin copolymer. In another embodiment, the structural layer is selected from a family of thermoplastic and thermoplastic vulcanized polymers.

In some embodiments, the outer layer is durable, thermoformable, chemical resistant, UV resistant, moisture resistant, impact resistant, scratch resistant, and has outdoor weatherability and soft feel.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 provides an illustration of a laminate with multiple layers.

FIG. 2 provides an illustration of a laminate with multiple layers for prosthetic functional decorative skin applications.

FIG. 3 provides an illustration of a laminate with multiple layers for outdoor furniture functional decorative skin applications.

FIG. 4 provides an illustration of a laminate with multiple layers for automotive cargo and trunk liners and floor mat functional decorative skin applications.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

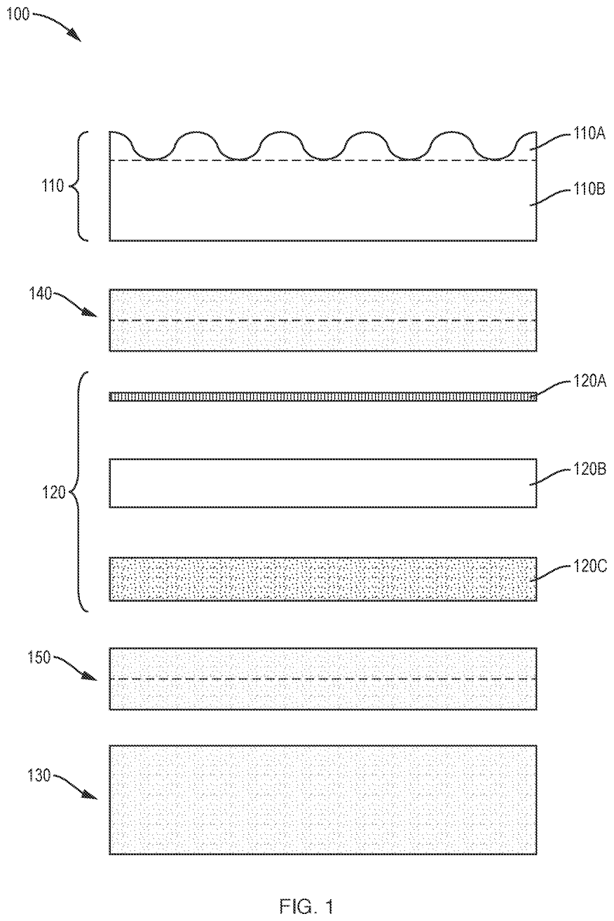

FIG. 1 is an illustration of a laminate 100 with multiple layers according to an embodiment of the present disclosure. As shown, the laminate 100 includes a surface layer 110, which also plays the role of a protective layer, a functional layer 120, which also plays the role of a decorative, graphics layer, and a structural layer 130, which also plays the role of a bottom layer. Two tie layers 140, 150 are represented as adhesive layers.

As shown, surface layer 110 includes an embossed pattern layer 110A as well as a print carrier layer 110B. The embossed pattern layer 110A can provide scratch and mar resistance, and be manufactured in process by roll or emboss paper. The print carrier layer 110B can be clear and washable, provide chemical resistance, scratch resistance, mar proof, and can be tinted or colored to provide aesthetic effects. The print carrier layer 110B can be formulated with various thermoplastic urethanes (TPU's) including copolyether-ester (COPE) and polyether-block-amide (COPA), with flame retardant additives.

Functional layer 120 includes an ink layer 120A, a print carrier layer 120B and a graphic effect layer 120C. The ink layer 120A can be a “skeleton foil” to transfer ink or digital print with open space for light to pass through printed on clear graphic film, and manufactured of a polyester material such as polyethylene terephthalate (PET). In combination with the print carrier layer 120B, the layers 120A, 120B may provide transfer print carrier graphic film based on PET, polycarbonate (PC) or acrylics graphic films. The graphic effect layer 120C can be a clear polymer film (e.g., polyester) with strontium-based luminescence additive and manufactured via a hot-melt process.

Structural layer 130 provides bulk physical properties on which layers 110, 120, 140, 150, among other materials, may be supported thereon. For example, the structural layer 130 can be formed of a PET substrate.

Tie layer 140 functions to provide adhesion between layers 110 and 120, while tie layer 150 functions to provide adhesion between layers 120 and 130. Both tie layers 140, 150 may be primed for direct printing, or formed as part of a hot-melt process, and manufactured using materials such as modified olefin, polyester, polyamide or TPU.

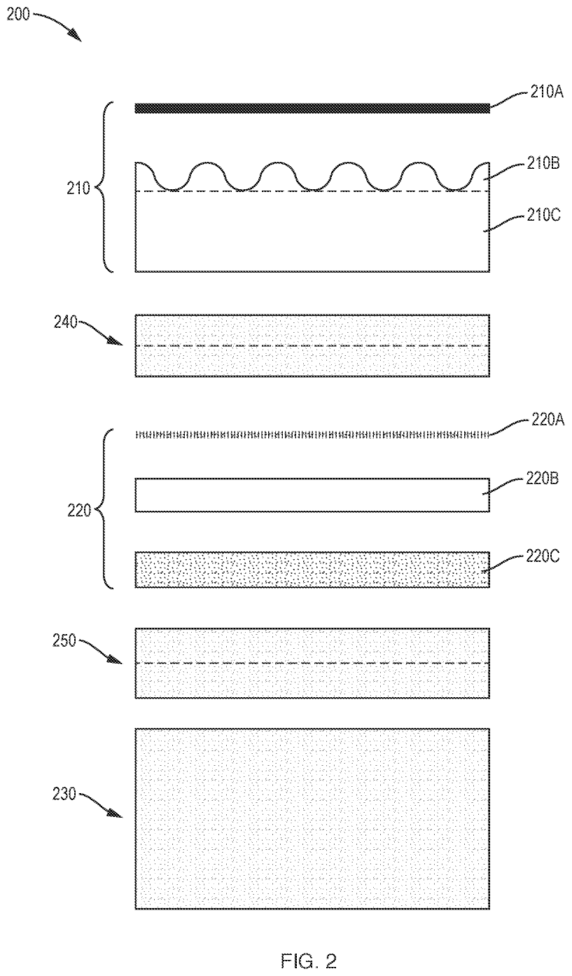

FIG. 2 is an illustration of a laminate 200 with multiple layers according to another embodiment of the present disclosure. In this embodiment, the laminate 200 may be useful in prosthetic functional decorative skin applications. The requirements of such applications include, without limitation, improved scratch and mar resistance, soft-durable haptic, high resolution decoration graphics, be thermo-formable, thermally insulating, and optionally be able to glow-in-the-dark for nighttime operation.

As shown, the laminate 200 illustrates a protective, surface layer(s) 210 having an aliphatic thermoplastic urethane (TPU) layer 210C along with other layers 210A, 210B. Another layer 220 represents the functional graphic layer(s) 220 having a thermal insulator based on TPU 220C along with other layers 220A, 220B. Another layer 230 represents the structural layer(s) 230 having a low temperature forming reinforced PET. The tie layer(s) 240, 250 are represented by adhesives based on hot melt or moisture cured polyurethane.

In one embodiment, the surface layer(s) 210 includes at least one protective surface layer 210C which plays the role of a protective layer 210C having about 3 mils thickness of at least one aliphatic thermoplastic urethane (TPU) layer 210C to provide UV block and absorbance, weather and chemical resistance, improved durability and cleanability, as well as a soft feel (e.g., soft-touch haptics) along with scratch and mar resistance.

The surface layer(s) 210 include additional layers such as a topcoat layer 210A of about 1 mil in thickness of optional moisture curable aliphatic TPU as a topcoat to provide UV and scratch resistance and mar resistance, as well as an emboss pattern layer 210B having a thickness of from about 2 to about 4 mils to provide a controlled gloss and reflective aesthetic, scratch and mar resistance.

The decorative layer(s) 220 include a graphics functional layer 220C having a thickness of from about 3 to about 4 mils of a thermal insulator material selected from at least one thermoplastic urethane (TPU) with fillers, blowing agents, combination of lightweight filler and blowing agent (e.g., Expancel® microspheres), or luminescent additives to provide insulation/lighting/luminescence effects (e.g., photoluminescent strontium aluminate). Such effects may reduce socket heating in the sun, and allow graphics and prosthetics to function in the dark. The decorative layer(s) 220 may include an ink printable layer 220A having a thickness of from about 0.2 to about 1 mil to provide a decoration with high resolution printed graphics (e.g., print can have “holes” to allow luminescent graphic effects “through”). The ink printable layer 220A may be formed of solvent and water-based pigments or dyes in polymer binder. Additionally, the decorative layers(s) 220 may include a print carrier graphic film layer 220B having a thickness of from about 1 to about 3 mils of print carrier graphic film (e.g., transparent) layer based on PET, PC or acrylic graphic film.

The bottom layer includes a structural layer(s) 230 having a low temperature forming reinforced polyester (PET) as a prosthetic socket for injection molded structural limb socket, and have a thickness in the range of from about 0.16 to about 0.20 inch.

Tie layer 240 functions to provide bonding between layers 210 and 220, while tie layer 250 functions to provide bonding between layers 220 and 230. Both tie layers 240, 250 may be formed of hot melt or moisture cured polyurethane, and have thicknesses of less than about 1 mil.

In one embodiment, the layers 210, 220, 240 and 250 collectively form a functional cap-stock laminate film, and in combination with the structural layer 230 forms an overall laminated product construction for prosthetic functional decorative skin applications.

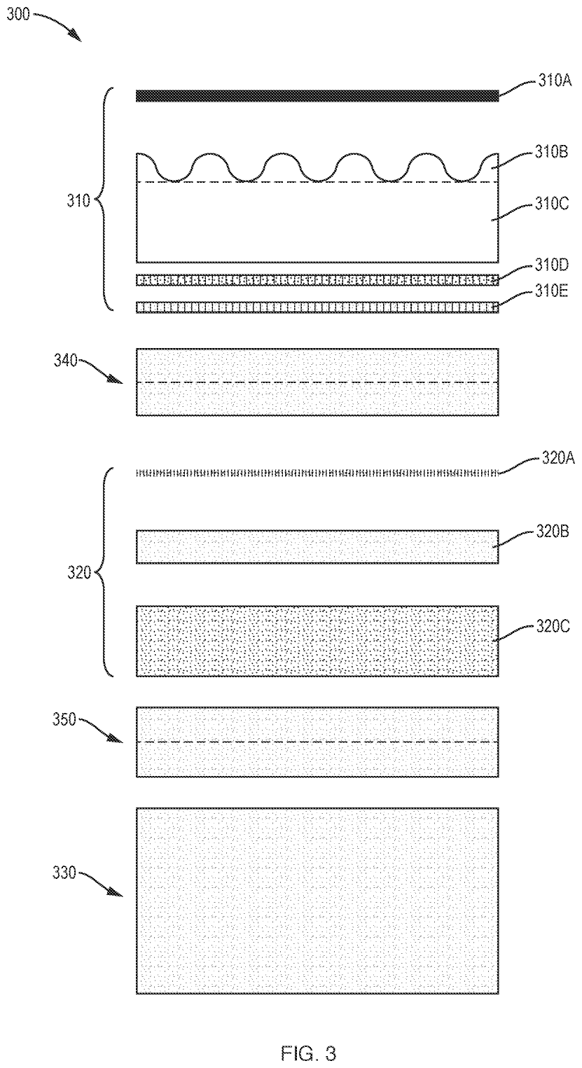

FIG. 3 is an illustration of a laminate 300 with multiple layers according to another embodiment of the present disclosure. In this embodiment, the laminate 300 may be useful in outdoor furniture functional decorative skin applications. The requirements of such applications include, without limitation, improved scratch and mar resistance, UV and weather resistance, high resolution decorative graphics, be electronically printable (e.g., solar cell, thermal resistance circuits), thermally insulating, and optionally, provide a solar-charging heated-seat surface skin.

The laminate 300 includes protective, decorative surface layer(s) 310 having a clear ionomer elastomer layer along with other layers, functional graphic layer(s) 320 having a thermal insulator layer based on ionomer with lightweight filler and/or blowing agents, and structural layer(s) 330 based on wood composite boards or plastics wood composites, among other layers that will be discussed in more details below. Tie layer(s) 340, 350 are represented by adhesives based on maleated functional olefin or clear maleic anhydride grafted EMA (ethylene methyl acrylate) copolymer (EMA-MAH), among others.

In one embodiment, the surface layer(s) 310 include a surface layer 310C which plays the role of a protective layer having about 2 to about 4 mils thickness of a clear ionomer elastomer material to provide UV (e.g., blocking/absorbing), weather and chemical resistance, cleanability and durability, as well as a soft feel (e.g., “soft-touch” haptics) along with scratch and mar resistance. Optionally, the surface layer 310C may include open wood grain aesthetic graphic.

In addition, the surface layer(s) 310 also has other layers such as about 1 mil thickness of moisture curable aliphatic TPU as a topcoat layer 310A to provide UV and scratch resistance, an emboss pattern layer 310B having a thickness of about 1 mil to provide a control gloss and reflective aesthetic (e.g., organic “sine” curve having rounded peak and valley cross-section), scratch and mar resistance, a hot melt layer 310D of about 1 mil thickness of clear maleated functional olefin, and an extrusion laminated skeleton print foil layer 310E of about 0.2 to about 0.5 mil thickness having a wood grain feature.

Decorative and graphic layer(s) 320 include a functional layer 320C having thickness in the range of from about 3 to about 4 mils of a thermal insulator material based on ionomer with lightweight filler and/or blowing agents with or without pigments to provide insulation, and an ink printable layer 320A having a thickness of about 1 to about 3 mils to provide decoration with high resolution printed graphics. The ink printable layer 320A may include a flexible ink printed graphic film for battery charging of photovoltaic or solar cells (e.g., perovskite, copper indium gallium selenide (CIGS), organic) or heating (e.g., printed flexible heating foil) applications, the functions based on position on outdoor furniture, e.g., seat versus arms of chair. In another embodiment, the decorative and graphic layer(s) 320 include a color pigmented maleic anhydride grafted EMA (ethylene methyl acrylate) copolymer (EMA-MAH) layer 320B of about 1 to about 3 mils in thickness to create aesthetic “read-through” color and barrier to under layers.

Structural layer(s) 330 can include wood composite board or plastic wood composite having a thickness of about 0.50 to about 1.0 inch which can be protected by the above laminate layers 310, 320, 340, 350. The structural layer(s) 330 may also function as a structural board for building furniture by supporting the above laminated layers 310, 320, 340, 350 to provide the desired protection, aesthetics and functions.

Tie layer 340 functions to provide bonding between layers 310 and 320, has a thickness of less than about 2 mils, and may be formed of a clear EMA-MAH encapsulant material. Tie layer 350 functions to provide bonding between layers 320 and 330, has a thickness of less than about 1 mil, and may be formed of hot melt maleated functional olefin homo and copolymers.

In one embodiment, the layers 310, 320, 340 and 350 collectively form a functional cap-stock laminate film, and in combination with the structural layer 330 forms an overall laminated product construction for outdoor furniture functional decorative skin applications.

In some embodiments, the laminate 200, 300 includes: (i) an inner layer 220, 320 having a design, (ii) an outer layer 210, 310 having additives, the outer layer 210, 310 having at least one of ionomer, ionomer blend, single cation ionomer, mixed cation ionomer, non-ionomer, and mixtures thereof, and the additives including at least one of non-silica hardener, siloxanes, amine and phenolic light stabilizers, and impact modifiers including copolymers of styrene-butadiene rubbers and acrylonitrile-butadiene-styrene copolymers. In this embodiment, the laminate 200, 300 further includes (iii) a tie layer 240, 340 between the inner layer 220, 320 and the outer layer 210, 310, and (iv) a structural layer 230, 330 configured to support (i), (ii) and (iii), and be protected by the same.

In one embodiment, the inner layer 220 further includes a plurality of layers, where the plurality of layers includes graphic carrier film layer 220A and 220B having decorative printed graphics, and a thermal insulator layer 220C selected from at least one of thermoplastic urethane with fillers, blowing agents, luminescent additives, and combinations thereof.

In one embodiment, the inner layer 320 includes a plurality of layers, where the plurality of layers includes a graphic carrier film layer 320A having a decorative printed graphics, a color pigmented maleic anhydride grafted ethylene methyl acrylate (EMA) copolymer layer 320B, and a thermal insulator layer 320C selected from at least one of ionomer with fillers, blowing agents, luminescent additives, and combinations thereof.

In one embodiment, the outer layer 210, 310 further includes a plurality of layers, where the plurality of layers includes: (i) a topcoat 210A, 310A of moisture curable aliphatic thermoplastic urethane, (ii) an embossed pattern layer 210B, 310B (iii) a clear layer of aliphatic thermoplastic urethane, or (iv) a combination of (ii) and (iii).

In one embodiment, the outer layer 310 includes a plurality of layers, where the plurality of layers includes: (i) a topcoat 310A of moisture curable aliphatic thermoplastic urethane, (ii) an embossed pattern layer 310B, (iii) a clear layer of ionomer elastomer 310C, (iv) a clear maleated functional olefin hot melt layer 310D, (v) a skeleton print foil with wood grain layer 310E, or (vi) a combination of (ii), (iii), (iv) and (v).

In some embodiments, the outer layer 310 can be durable, thermoformable, chemical resistant, UV resistant, moisture resistant, impact resistant, scratch resistant, and has outdoor weatherability and soft feel.

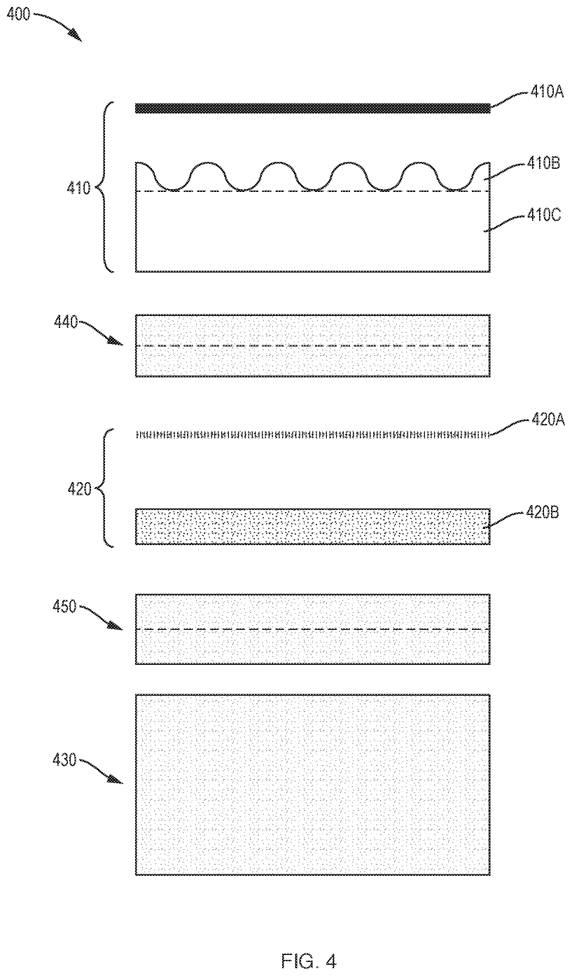

FIG. 4 is an illustration of a laminate 400 according to another embodiment of the present disclosure. In this embodiment, the laminate 400 with multiple layers may be useful in both injection molded and thermoformed skins for automotive cargo, trunk liners and floor mat functional decorative skin applications. The requirements for such applications include, without limitation, improved dirt, mud, water, scratch and mar resistance, “tough rubber” haptic, high-resolution decorative graphics, be thermo-formable, and optionally provide luxury aesthetics and colors.

As shown, the laminate 400 illustrates the protective, surface layer(s) 410 having an ionomer elastomer layer 410C with nano-silica or pearlescent pigments along with other layers 410A, 410B. Another layer 420 represents the functional graphic layer(s) 420 having an ionomer elastomer layer 420B with solid pigment along with other layers 420A. Another layer 430 represents the structural layer(s) 430 based on thermoplastic elastomer or thermoplastics vulcanized materials along with other layers. The tie layer(s) 440, 450 are represented by adhesives based on maleated functional olefins.

In one embodiment, the surface layer(s) 410 illustrates the protective surface layer(s) 410C of about 3 mils thickness of an ionomer elastomer layer with at least one nano-silica and at least one pearlescent pigment (e.g., calcining mica coated with a metal oxide) to provide UV (blocking/absorbing), weather (e.g., moisture, mud) and chemical resistance, cleanability, “durable rubber” haptics, enhanced grip and anti-slip properties, as well as a soft feel along with scratch and mar resistance.

In addition, the surface layers(s) 410 also has other layers such as layer 410A having about 1 mil thickness of moisture curable aliphatic TPU as a topcoat to provide UV and scratch resistance, as well as an emboss pattern layer 410B having a thickness of from about 2 to about 4 mils to provide a control gloss and reflective aesthetic (e.g., organic “sine” curve having rounded peak and valley cross-section), scratch and mar resistance.

In some embodiments, the surface layer 410 may include ionomer, ionomer blend, single cation ionomer, mixed cation ionomer, non-ionomer, and mixtures thereof. Examples of ionomer include without limitation copolymers of ethylene with acrylic and methacrylic co-monomers optionally with alkyl acrylate or methacrylate ter-monomer, and combinations thereof. Examples of non-ionomer include without limitation polyurethanes, polyurethane blends, polyamides, acrylics, acrylics blends, polyester, polyester blends, polyester copolymers, polyester copolymer blends, and combinations thereof.

In some embodiments, the surface layer 410 may include additives including at least one of non-silica hardener, siloxanes, amine and phenolic light stabilizers, and impact modifiers including copolymers of styrene-butadiene rubbers and acrylonitrile-butadiene-styrene copolymers.

In some embodiments, the surface layer 410 may be configured to exhibit at least one of the following properties: luminescence, thermochromism, textured look, optics enabled, and anti-static. The surface layer 410 may be further configured to provide an aesthetic effect, the aesthetic effect selected from at least one of fluorescence, pearlescence, stone-look appearance and wood-look appearance.

The decorative layer(s) 420 include a graphic functional layer 420B having a thickness of about 3 to about 4 mils of an ionomer elastomer and a solid pigment for print-design contrast and opaque barrier to inexpensive backing product, along with another layer 420A having a thickness of from about 0.2 to about 1 mil of reverse printed ink onto “adhesive” to provide decoration with high-resolution printed graphic. The decorative layer(s) 420 functions to provide decorative effects, including without limitation, blocking “read-through” of TPE/TPV layer while adding additional depth/luster, and printed graphics with “holes” to allow lower colors/graphics effects “through.”

In some embodiments, the functional layer 420 includes at least one of polyurethanes, polyamides, polyacrylates, polyureas, polyimides, olefinic copolymers, terpolymers, functionalized olefins, and combinations thereof.

The bottom layer includes a structural layer(s) 430 formed from a thermoplastics elastomer (TPE) or thermoplastic vulcanized (TPZ) material(s) (e.g., PP+EPDM blend, urethane, EVA) having a thickness of about 0.100 to 0.250 inch which can be protected by the above laminate layers 410, 420, 440, 450 to provide aesthetics in durable rubber mat or liner. The structural layer(s) 430 may function as “rubber sheet” product base, which be made via injection molded or extrusion laminated elastomer base for creating body of tough rubber mat or liner product.

Tie layer(s) 440 functions to provide bonding between surface layer 410 and functional layer 420, while tie layer 450 functions to provide bonding between functional layer 420 and structural layer 430. Both tie layers 440, 450 may be formed of maleic anhydride grafted (MAH) functional olefin copolymer (EVA) each having a thickness of less than about 1 mil.

In some embodiments, the tie layers 440, 450 include at least one of grafted homo or co-polymers of ethylene, propylene, or butene, each optionally mixed with maleic anhydride, maleic acid, or glycidyl methacrylate. In some embodiments, the tie layers 440, 450 include at least one of olefinic, non-olefinic, thermoplastic adhesive, and combinations thereof. Examples of thermoplastic adhesive includes without limitation homo or copolymers or terpolymers of olefinic polymers grafted with maleic anhydride or glycidyl acrylate or methacrylate or amine groups or polyurethanes or polyamides or epoxy resins.

In one embodiment, the layers 410, 420, 440 and 450 collectively form a functional cap-stock laminate film, and in combination with the structural layer 430 forms an overall laminated product construction for automotive cargo, trunk liners and floor mat functional decorative skin applications.

In one embodiment, a laminate 400 includes a structural layer 430, an inner layer 420 having a design, a first tie layer 450 between the structural layer 430 and the inner layer 420, an outer layer 410 having additives, the outer layer having at least one of ionomer, ionomer blend, single cation ionomer, mixed cation ionomer, non-ionomer, and mixtures thereof, and the additives having at least one of non-silica hardener, siloxanes, amine and phenolic light stabilizers, and impact modifiers including copolymers of styrene-butadiene rubbers and acrylonitrile-butadiene-styrene copolymers, and a second tie layer 440 between the inner layer 420 and the outer layer 410.

In one embodiment, the inner layer 420 further includes a plurality of layers, where the plurality of layers 420 is selected from the group consisting of a graphic carrier film layer 420A having a decorative printed graphics, and a decorative layer 420B having one or more ionomers with solid color pigment.

In one embodiment, the outer layer 410 further includes a plurality of layers, where the plurality of layers 410 is selected from the group consisting of (i) a topcoat 410A of moisture curable aliphatic thermoplastic urethane, (ii) an embossed pattern layer 410B, (iii) a clear layer 410C selected from the group consisting of ionomer elastomer, nano silica and pearlescent pigment, or (iv) a combination of (ii) and (iii).

In some embodiments, the laminates or laminate coverings as described herein can be durable, thermoformable, chemical resistant, UV resistant, moisture resistant, impact resistant, scratch resistant, and has outdoor weatherability and soft feel.

Provided in yet another aspect of the instant invention is a process of manufacturing the laminate, said process selected from tandem extrusion, coextrusion, belt lamination, roll coating, and hot melt lamination or combinations thereof. In some embodiments, the laminate or laminate covering may be manufactured as an extrudable composition, which is meant to represent a composition that can manufactured by an extrusion process known to one skilled in the art. Furthermore, an extrudable composition represents a family of homo and copolymers that possess required melt viscosity so that those polymers are extrudable in nature.

DEFINITIONS—The terms included in the instant specification to describe the invention have the general meaning as understood by one skilled in the art in addition to the discussion below.

The term “laminate” or “laminate covering” as used herein is intended to represent a sheet comprising a single or multiple layers of materials put together in a composite form or wherein the multiple layers are glued together using an adhesive tie layer. The laminate can be used to cover surfaces such as floors, walls, cabinets, consumer goods, durable goods, and the like, and is intended to be strong, durable, provide sound insulation, improved appearance, moisture, chemical resistant, and improved weatherability.

The term “surface layer” is intended to represent the top layer of the laminate wherein the top layer is what is exposed to moisture, wear and tear, can be transparent, soft, hard, and is intended to provide strength, durability, sound insulation, attractive appearance, tactile feel, moisture and chemical resistance, and improved weatherability.

The term “functional layer” as used herein generally represents an inner layer of the laminate and is functional wherein it provides stability for the laminate, has aesthetic effects such as fluorescence, luminescence, pearlescence, stone look, wood look, and other decorative or functional properties. It is understood that some functionality can also be applied to surface, intermediate, or structural layers as performance and aesthetically required.

The term “tie layer” is intended to represent a layer, or series of compatibilized layers, that generally functions as a layer with adhesive properties and, for example, functions to join a surface layer and a functional layer, or a functional layer and a structural layer, even if the layers are otherwise incompatible with each other in terms of being bound together. It is understood that there can be one or more tie layers within a laminate or laminate covering.

The term “structural layer” is intended to describe the layer of the laminate or laminate covering which, in a general sense, comes in contact with the intended surface, such as a floor, wall, cabinet, and the like, that is to be covered by the laminate or laminate covering.

The term “aesthetic effect” is intended to describe an effect which is generally associated with the look and feel of a material and has more of a decorative and appeal function. Illustrative examples of an aesthetic effect include surface texture, surface feel, surface optics, printed graphics and the like.

The term “ionomer(s)” as used herein is intended to represent chemical entities of a class of synthetic ethylene-based thermoplastic resins consisting of a copolymer of ethylene with acid containing co-monomers wherein some or all of the acid groups are neutralized by suitable cations to provide ionic cross-links. Illustrative examples of an ionomer are ethylene-acrylic or methacrylic acid copolymers neutralized with metal cations or ethylene-acrylic or methacrylic acid-alkyl acrylate copolymers neutralized with metal cations. Surlyn® and Iotek® are examples of some of commercially available ionomers. Disclosed embodiments therein may also include other ionomer materials in “Introduction to Ionomers” by Adi Eisenberg and Joon-Seop Kim published in 1998 and available at https://pubs.acs.org/doi/10.1021/ja985693m.

As used herein, a carboxylic acid group neutralized by a metal cation is intended to represent a carboxylic acid (COOH) group which is neutralized by a metal base to form a (COO-M+) moiety wherein M+ represents a metal cation. Illustrative examples of a metal base are lithium hydroxide, sodium hydroxide and zinc oxide. Illustrative examples of a metal cation are Li, Na, K, Zn, Mg and Na.

The terms “single cation ionomer(s)” and “mixed cation ionomer(s)” as used herein are intended to represent lithium or sodium or zinc neutralized ethylene-acrylic or methacrylic acid ionomers for single cation ionomers and blends of lithium/sodium or lithium/zinc or sodium/zinc neutralized ethylene-acrylic or methacrylic acid ionomers for mixed cation ionomers.

The term “polyamide(s)” as used herein is intended to represent chemical entities of a class of polyamide(s) including without limitation homopolymers of PA11 (polyamide 11), homopolymers of PA12 (polyamide 12), copolymers and terpolymers of PA6 (polyamide 6), copolymers and terpolymers of PA10 (polyamide 10), copolymers and terpolymers of PA12, copolymers and terpolymers of PA 6/6, copolymers and terpolymers of PA 6/12. Thermoplastic polyamides urethanes as mentioned in the “Nylon Plastics Handbook” by Melvin I. Kohan published in 1995 and available at https://pubs.acs.org/doi/10.1021/ja9655808 are incorporated herein by reference. Furthermore, polyamides with ionomer blends are intended to represent PA11 or PA12 or PA6 blended with zinc neutralized ethylene-acrylic or methacrylic acid ionomers.

The terms “acrylic(s)” and “acrylic blend(s)” as used herein are intended to include classes of acrylics as illustrated by polyacrylates, polymethylmethacrylates, copolymers of alkyl, among others, as described at https://en.wikipedia.org/wiki/poly(methyl methacrylate) and https://en.wikipedia.org/wiki/Acrylate polymer.

The term “thermoplastic urethane(s)” as used herein is intended to represent chemical entities of a class of the aromatic or aliphatic diisocyanate with aliphatic or aromatic diols or polyols. Thermoplastic polyurethanes (TPU's) as mentioned in the “Szycher's Handbook of Polyurethanes” by Michael Szycher published in 2012 and available at https://www.routledgehandbooks.com/doi/10.1201/b12343 are incorporated herein by reference.

The term “aliphatic homo and copolymers of polyesters” is intended to represent chemical entities of a class of homo and copolyesters as outlined in the Handbook of Thermoplastic Polyesters: Homopolymers, Copolymers, Blends, and Composites by Fakirov (Editor), 2002 https://onlinelibrary.wiley.com/doi/book/10.1002/3527601961.

The term “polycarbonates” as used herein represents chemical entities of a class of polycarbonates as outlined in the Handbook of Polycarbonate Science and Technology by the editor John T. Bendler, 1999. https://books.google.com/books/about/Handbook_of_Polycarbonate_Science_and_Te.html?id=Y L-cza 44N8C.

The term “plastics additives and impact modifiers” as used herein is intended to include a class of acrylonitrile-butadiene-styrene (ABS) or methacrylate-butadiene-styrene (MBS) as outlined in the Plastics Additives and Modifiers Handbook by Jesse Edenbaum, 1992 https://books.google.com/books/about/Plastics_Additives_and_Modifiers_Handboo.html?id=R8 FTAAAAMAAJ.

The term “additive” as used herein is intended to represent a family of materials added to another substance or product to produce specific properties in the combined substance or final products. For example, the addition of nano-silica or siloxane to ionomers of the present invention enhances shear resistance, or abrasion resistance, or a combination of both. Additives could be either, both or all, non-silica hardener, siloxanes, and/or impact modifiers. Amine and phenolic light stabilizers may also be an additive.

The term “impact modifiers” could also include the use of other polymeric impact modifier chemistries including low glass transition material additives, thermoplastic elastomers (TPE's) and rubbers, functional olefin polymers, ethylene copolymers, and core-shell modifiers.

The terms “nano-silica” and “siloxane(s)” as used herein are independently intended to represent chemical entities of a class of silicone or siloxane-based material as outlined in the “Handbook of Silicon Based MEMS Materials and Technologies” by Markku Tilli et al. published in 2010 and available at https://www.sciencedirect.com/book/9780815515944/handbook-of-silicon-based-mems-materials-and-technologies. Additionally, the foregoing terms represent certain amount of additives including nano-silica and siloxane additives that are dispersed in one or more carrier resins that are compatible with matrix polymers for the laminate compositions.

As used herein, “acrylic and methacrylic co-monomers” represent a class of acid comonomers. Illustrative examples are acrylic acid and methacrylic acid.

As used herein, “alkyl acrylate and methacrylate ter-monomer” represents a class of third monomer to produce copolymers having three monomers together like in the case of copolymer of ethylene with acrylic or methacrylic acid and methyl acrylate or butyl acrylate. Illustrative examples include methyl acrylate or butyl acrylate.

The terms “scratch resistant” (or abrasion resistant) are intended to describe the ability of a material/surface to resist various types of damage such as scratches, gouges, wear, and other physical flaws. This parameter is crucial in the coating industry since the degree of abrasion and scratch resistance in coatings can govern the longevity of the product and also determine its ability to protect the coated material against corrosion.

While the present invention has been described with reference to certain embodiments thereof, it should be understood by those skilled in the art that various changes may be made and equivalents may be substituted without departing from the true spirit and scope of the invention. In addition, many modifications may be made to adapt to a particular situation, indication, material and composition of matter, process step or steps, without departing from the spirit and scope of the present invention. All such modifications are intended to be within the scope of the present invention.

1. A laminate covering comprising:
a functional layer including a printable layer disposed over a graphic effect layer;
an optics enabled surface layer having additives, the surface layer comprising at least one of ionomer, ionomer blend, single cation ionomer, mixed cation ionomer, non-ionomer, and mixtures thereof, and the additives comprising at least one of non-silica hardener, siloxanes, amine and phenolic light stabilizers, and impact modifiers including copolymers of styrene-butadiene rubbers and acrylonitrile-butadiene-styrene copolymers; and
a tie layer between the functional layer and the surface layer.

2. The laminate covering of claim 1, wherein the functional layer comprises polyurethanes, polyamides, polyacrylates, polyureas, polyimides, olefinic copolymers, terpolymers, functionalized olefins, or a combination thereof.

3. The laminate covering of claim 1, wherein the ionomer is selected from the group consisting of copolymers of ethylene with acrylic and methacrylic co-monomers optionally with alkyl acrylate and methacrylate ter-monomer, and combinations thereof, and wherein the non-ionomer is selected from the group consisting of polyurethanes, polyurethane blends, polyamides, acrylics, acrylic blends, polyester, polyester blends, polyester copolymers, polyester copolymer blends, and combinations thereof.

4. The laminate covering of claim 1, wherein the tie layer comprises at least one of grafted homo or co-polymers of ethylene, propylene, or butene, each optionally mixed with maleic anhydride, maleic acid, or glycidyl methacrylate.

5. The laminate covering of claim 1, wherein the tie layer comprises at least one of olefinic, non-olefinic, thermoplastic adhesive, and combinations thereof.

6. The laminate covering of claim 5, wherein the thermoplastic adhesive is selected from the group consisting of homo or copolymers or terpolymers of olefinic polymers grafted with maleic anhydride or glycidyl acrylate or methacrylate or amine groups or polyurethanes or polyamides or epoxy resins.

7. The laminate covering of claim 1, wherein the thickness of the functional layer is in the range of from about 1 mil to about 25 mils, wherein the thickness of the surface layer is in the range of from about 0.1 mil to about 20 mils, and wherein the thickness of the laminate covering is in the range of from about 0.1 mm to about 5 mm.

8. The laminate covering of claim 1, wherein the surface layer is configured to exhibit at least one of the following properties: luminescence, thermochromism, textured look via the optics enabling, and anti-static, and wherein the surface layer is further configured to provide an aesthetic effect, the aesthetic effect selected from at least one of fluorescence, pearlescence, stone-look appearance and wood-look appearance.

9. The laminate covering of claim 1, wherein the surface layer is durable, thermoformable, chemical resistant, UV resistant, moisture resistant, impact resistant, scratch resistant, and has outdoor weatherability and soft feel.

10. A process of manufacturing the laminate covering of claim 1, wherein the process is selected from the group consisting of tandem extrusion, coextrusion, belt lamination, roll coating and hot melt lamination.

11. A laminate comprising:
(i) an inner layer having a design provided by an inclusion of a printable layer disposed over a graphic effect layer;
(ii) an optics enabled outer layer having additives, the outer layer comprising at least one of ionomer, ionomer blend, single cation ionomer, mixed cation ionomer, non-ionomer, and mixtures thereof, and the additives comprising at least one of non-silica hardener, siloxanes, amine and phenolic light stabilizers, and impact modifiers including copolymers of styrene-butadiene rubbers and acrylonitrile-butadiene-styrene copolymers;
(iii) a tie layer between the inner layer and the outer layer; and
(iv) a structural layer configured to support (i), (ii) and (iii).

12. The laminate of claim 11, wherein the inner layer further comprises a plurality of layers, wherein the plurality of layers includes one or more of:
a graphic carrier film layer having a decorative printed graphics; and
a thermal insulator layer selected from at least one of thermoplastic urethane with fillers, blowing agents, luminescent additives, and combinations thereof.

13. The laminate of claim 12, wherein the outer layer further comprises a plurality of layers, wherein the plurality of layers is selected from the group consisting of:
(i) a topcoat of moisture curable aliphatic thermoplastic urethane;
(ii) an embossed pattern layer;
(iii) a clear layer of aliphatic thermoplastic urethane; or
(iv) a combination of (ii) and (iii).

14. The laminate of claim 13, wherein the tie layer includes hot melt or moisture cured polyurethane.

15. The laminate of claim 13, wherein the structural layer is an injection molded structural limb socket formed of a low temperature reinforced thermoplastic polyester (PET) compound.

16. The laminate of claim 11, wherein the inner layer further comprises a plurality of layers, wherein the plurality of layers includes one or more of:
a graphic carrier film layer having a decorative printed graphics;
a color pigmented maleic anhydride grafted ethylene methyl acrylate (EMA) copolymer; and
a thermal insulator layer selected from at least one of ionomer with fillers, blowing agents, luminescent additives, and combinations thereof.

17. The laminate of claim 16, wherein the outer layer further comprises a plurality of layers, wherein the plurality of layers is selected from the group consisting of:
(i) a topcoat of moisture curable aliphatic thermoplastic urethane;
(ii) an embossed pattern layer;
(iii) a layer of ionomer elastomer;
(iv) a clear maleated functional olefin hot melt layer;
(v) a skeleton print foil with wood grain layer; or
(vi) a combination of (ii), (iii), (iv) and (v).

18. The laminate of claim 17, wherein the tie layer includes hot melt maleated olefin homo and copolymers.

19. The laminate of claim 17, wherein the structural layer is selected from the group consisting of wood composite boards and plastic wood composites.

20. The laminate of claim 11, wherein the outer layer is durable, thermoformable, chemical resistant, UV resistant, moisture resistant, impact resistant, scratch resistant, and has outdoor weatherability and soft feel.

21-26. (canceled)
